(19) United States
(12) Patent Application Publication
(10) Pub. No.: US 2025/0194969 A1
(43) Pub. Date: Jun. 19, 2025

(54) A PHOTOPLETHYSMOGRAPY SENSOR HAVING A NOVEL ARRANGEMENT

(71) Applicant: WELL BEING DIGITAL LIMITED, Pak Shek Kok, New Territories (HK)

(72) Inventor: Ming Yip Wallace WONG, Pak Shek Kok, New Territories (HK)

(21) Appl. No.: 18/846,649

(22) PCT Filed: Mar. 23, 2023
(86) PCT No.: PCT/CN2023/083487

§ 371 Date: Sep. 12, 2024

(30) Foreign Application Priority Data
Mar. 28, 2022 (HK) 32022050725.7

(51) Int. Cl. A61B 5/1459; A61B 5/00

(52) U.S. Cl. CPC A61B 5/1459; A61B 5/6817; A61B 2562/0238; A61B 2562/043

(57) ABSTRACT

A PPG sensor that comprises two emitters each emitting in a different wavelength and at least one optical sensor. The emitters are placed to emit through a body part of the user to the optical sensor in the same direction. However, the emitter of the shorter wavelength is placed further from the optical sensor, so as to force the emission to travel deeper into the tissue before reaching the optical sensor. This resolves the natural difference in penetration depths of the two wavelengths, especially if the two wavelengths are so far apart on the electromagnetic spectrum that their respective absorption mechanisms are different.

### FIELD OF INVENTION

The invention relates to the field of wearable monitors. In particular, the invention relates to photoplethysmography sensors.

### BACKGROUND OF THE INVENTION

Photoplethysmogram (PPG) sensors are wearable devices that transmit light into the flesh of a subject to detect the amount of light transmitted or absorbed. Hence, PPG sensors can be thought of as crude forms of spectrometers which are applied onto the body. PPG sensors used in wearable devices tend to be used indiscriminately of specific blood components, that is, the whole body of blood is measured instead of a specific analyte. The result is a crude sinusoidal signal that correlates to changes in volume of blood inside the flesh, which shows the pulse of the subject.

However, some PPG sensors can measure specific analytes in blood by transmitting light of specific wavelengths which the specific analytes respond to. For example oximeters transmit both red light and infrared light through a finger, which are absorbed by oxygenated haemoglobin (bright red) and deoxygenated haemoglobin (dark red or blue) respectively. It is possible to quantify the extent of oxygenation by comparing the absorption readings of the two wavelengths.

However, transmission oximeters can only be used on thin body parts such as the fingers or earlobes. This is because the body part must have substantial blood flow and a relatively low range of muscular activity that could cause sudden blood content change within the body part. Also, thicker body parts such as the biceps, thighs and wrists tend to absorb so much of the light from the PPG sensor that there is no transmission.

It has been proposed that the wall of the ear canal meets the requirements of a good, in vivo, PPG application site using reflective PPG. Reflective PPG sensors transmit light into tissue and measure the amount of light that is scattered back to see how much of the light has been absorbed.

FIG. 1 illustrates a PPG sensor according to the prior art, taken from https://www.researchgate.net/publication/288165900_Investigation_of_photoplethysmograph y_and_arterial_blood_oxygen_saturation_from_the_ear-canal_and_the_finger_under_conditions_of_artificially_induced_hypothermia. FIG. 2 is a schematic diagram explaining the prior art of FIG. 1.

The prior art is in the form of a nozzle that can be inserted into the ear canal. The nozzle is almost round in the cross-section, except that a portion of the round shape is hived off to provide a flat surface. Situated on the flat surface of are two emitters 101 and an optical sensor 103. The two emitters 101 are arranged side by side, and to one side of the optical sensor 103 as shown in FIG. 2, which is the plan view of the flattened portion of the nozzle of FIG. 1. The straight arrows in FIG. 2 illustrates how light from each of the emitters is detected by the optical sensor upon being scattered by the issue defining the ear canal, i.e. the ear canal wall.

The two emitters 101 take turns to emit light of a different wavelength into the wall of the ear canal. The light is scattered by the tissue, such that some of the light reaches the optical sensor 203. The switch-over rate between the two emitters is very fast and is in terms of micro-seconds, such that the single optical sensor would appear to be reading the absorption in both wavelengths simultaneously.

The wavelength of light emitted by one emitter can be absorbed by tissue components not including the target analyte. The wavelength of light emitted by the other emitter can be absorbed by tissue components including the target analyte. The difference in absorption readings can be attributed to the amount of the target analyte in the body.

The trajectories of the emissions through the ear canal wall are different due to different scattering and absorption behaviour of the different wavelengths. Light with shorter wavelength tends to travel less deeply into the tissue. This means that light of the two different wavelengths may pass through different volumes or amounts of tissues components. If the difference is significant, the absorption readings obtained in the two wavelengths cannot be compared meaningfully. Hence, PPG sensors, even the afore-described oximeters, are limited to using only similar wavelengths, or wavelengths which are near to each other on the electromagnetic spectrum, and the locations of the emitters has to be placed very close to each other to avoid huge difference in trajectories (it is a basic spectrometric requirement that amounts of tissues components should be the same for both the wavelengths so that the absorption readings can be compared.)

Moreover, the nozzle in FIG. 1 has to be long enough to accommodate the linear arrangement of the emitters and optical sensor. Unfortunately, a long nozzle in the ear can be uncomfortable and unsafe to the user.

Accordingly, it is desirable to propose PPG devices that provide a possibility of applying light of in wavelengths that are very different, or significantly far apart, on the electromagnetic spectrum. Furthermore, it is also desirable to propose ear worn PPG devices that can be more comfortable to wear.

### STATEMENT OF INVENTION

In a first aspect, the invention proposes a PPG sensor comprising an earbud nozzle that can be inserted into the ear canal of a user; the nozzle having a curved surface; a first emitter, a second emitter and an optical sensor arranged on the curved surface of the nozzle; such that the first emitter and the second emitter are capable of emitting into the wall of the ear canal; the optical sensor is capable of monitoring the light from the wall of the ear canal; wherein the first emitter is placed on a first side on the curvature of the curved surface; the second emitter is placed on a second side on the curvature of the curved surface; the optical sensor is placed on third side on the curvature of the curved surface; and the first emitter and the second emitter are placed to one side of the optical sensor on the curvature of the curved surface.

Accordingly, the invention provides several possible advantages. Firstly the emitters are not arranged along the length of the nozzle with the optical sensor to form a row. This provides the possibility of a shorter nozzle which is more comfortable to the wear of the earbud. Secondly, the invention provides the possibility that the emitters are placed away from the optical sensor by the same angular difference, ensuring almost identical or similar trajectories through the ear canal wall.

Preferably, the first emitter emits light of a first wavelength; the second emitter emits light of a second wavelength; the first wavelength being shorter than the second wavelength; and the first emitter being further on the curvature of the curved surface from the optical sensor than the second emitter.

As shorter wavelengths do not penetrate as deeply as longer wavelengths, placing the emitter of the shorter wavelength from the optical sensor requires the emission of the shorter wavelength to travel deeper in the ear canal tissue before sufficient back scattering of the emission reaches the optical sensor. This increases the likelihood that the trajectory of the shorter wavelength is more coincident, or overlaps more significantly with the trajectory of the longer wavelength. This arrangement provides that two far apart wavelengths may be used in together to monitor blood analytes in the same PPG sensor, which was not possible in the prior art PPG sensors. For example, one wavelength may be an infrared wavelength while the other a green wavelength.

Optionally, first wavelength and the second wavelength are selected such that the absorption mechanism of first wavelength by a target analyte in the wall of the ear canal is different from the absorption mechanism of the second wavelength by the target analyte.

Optionally, the first emitter and the second emitter arranged into a row of emitters; the row of emitters being placed on the same side on the curvature of the curved surface. In this case, the first emitter and the second emitter are on the same point on the curvature of the curved surface.

Preferably, the optical sensor is a first optical sensor, the PPG sensor further comprising a second optical sensor; the second optical sensor arranged on the curved surface of the nozzle; such that the second optical sensor is capable of monitoring the light from the wall of the ear canal; wherein the first emitter and the second emitter are placed to one side of the second optical sensor on the curvature of the curved surface.

This provides the possibility of a PPG sensor having two emitters and two optical sensors, but without requiring the length of the earbud nozzle to accommodate all four emitters and optical sensors long the length of the earbud nozzle.

Preferably, the first optical sensor is capable of monitoring light of the first wavelength; the second optical sensor is capable of monitoring light of the second wavelength; the first emitter, the second emitter, the first optical sensor and the second optical sensor arranged such that: when in the PPG sensor is in use, a first trajectory of light in the wall of the ear canal between the first emitter and the first optical sensor overlaps with a second trajectory of light in the wall of the ear canal between second emitter and the second optical sensor.

This increases the likelihood that the trajectories between each of the two emitter-and-sensor pair overlap significantly.

More preferably, the first trajectory and the second trajectory cross.

In a second aspect, the invention provides a PPG sensor comprising a substrate, the substrate having a surface for being placed against a body part of a user; the surface of the substrate provided with: a) first emitter for emitting light of a first wavelength into the body part, b) second emitter for emitting light of a second wavelength into the body part, and c) at least one optical sensor for sensing light coming from within the body part; the first emitter and the second emitter arranged on the same side of the at least one optical sensor, such that the first emitter emits into a first position in the body part; the second emitter emits into a second position in the body part; wherein the trajectory of light from the first position to the at least one optical sensor is greater than the trajectory of light from the second position to the at least one optical sensor; and the first wavelength is shorter than the second wavelength.

For example, the substrate can be part of a mouth guard for placement against the floor of the mouth. This provides an alternative site to the ear canal wall for blood analyte monitoring.

There are many ways to provide that one trajectory is greater than the other, such as by the angle of emission.

Therefore, optionally, the first emitter emits into a first position in the body part by pointing in a first emission direction in the body; the second emitter emits into a second position in the body part by pointing in a second emission direction in the body; and the at least one optical sensor is pointed in a sensing direction to sense light coming from within the body part in a sensing direction; wherein the angle between the first emission direction and the sensing direction is greater than the angle between the second emission direction and the sensing direction.

Possibly, the angle between the first emission direction and the sensing direction is divergent. This feature means that being divergent with the sensing direction is optional for both emission directions, as long as the trajectories overlap significantly. More so, this feature shows that the first emission direction is divergent with the sensing direction, while not requiring the second emission direction to be divergent with the sensing direction. Hence, optionally,

Preferably, however, the angle between the second emission direction and the sensing direction is divergent

Typically, the surface of the substrate is convex; the curvature of the convex surface providing the divergence.

In a further aspect, the invention provides a PPG sensor comprising a substrate, the substrate having a surface for being placed against a body part of a user; the surface of the substrate provided with: a) a first optical sensor for monitoring light of a first wavelength from the body part; b) a second optical sensor for monitoring light of a second wavelength from the body part; c) at least one emitter for emitting light into the body part in the first wavelength and in the second wavelength, in an emission direction; the first optical sensor and the second optical sensor arranged on the same side of the at least one emitter, such that the first optical sensor monitors light in a first position in the body part; the second optical sensor for monitors light in a second position in the body part; the trajectory of light from the first position to the at least one emitter is greater than the trajectory of light from the second position to the at least one optical sensor; and the first wavelength is shorter than the second wavelength.

Optionally, the first position is provided as a first sensing direction; the second position is provided as a second sensing direction; the angle between the first sensing direction and the emission direction is greater than the angle between the second sensing direction and the emission direction to provide that the trajectory of light from the emitter to the first optical sensor is longer than the trajectory of light from the emitter to the second optical sensor. This feature means that being divergent with the emission direction is optional for both emission directions, as long as the trajectories overlap significantly. More so, this feature shows that the first sensing direction is divergent with the emission direction while not requiring the second sensing direction to be divergent with the emission direction. Hence, optionally,

Therefore, preferably, the angle between the first sensing direction and the emission direction is divergent. More preferably, the angle between the second sensing direction and the emission direction is divergent.

Typically, surface of the substrate is convex; the curvature of the convex surface providing the divergence.

In a further aspect, the invention provides a PPG sensor for being placed against a body part, comprising at least a first emitter; at least a first optical sensor; the first emitter configured to emit in a direction into the body part that forms a divergent angle with the direction in which the first optical sensor is configured to monitor light form the body part.

This feature covers embodiments with a flat surface for placing against a relatively flat body part, and the angular emission direction of the first emitter provides for the longer trajectory than directing emission into the body part orthogonally to the surface of the body part.

Preferably, the PPG sensor further comprises a second emitter; the second emitter configured to emit in a direction into the body part that forms a divergent angle with the direction in which the first optical sensor is configured to monitor light form the body part.

More preferably, the PPG sensor further comprises: a second optical sensor; the first emitter configured to emit in a direction into the body part that forms a divergent angle with the direction in which the second optical sensor is configured to monitor light form the body part.

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 shows prior art for a comparative example to the embodiment of FIG. 3;

FIG. 2 illustrates the workings of the prior art of FIG. 1;

FIG. 3 shows an embodiment of the invention;

FIG. 4 illustrates a part of the embodiment of FIG. 3 schematically;

FIG. 5 illustrates functional modules that may be provided in the embodiment of FIG. 3;

FIG. 6 is a cross-sectional side view of the part shown in FIG. 4, during operation;

FIG. 7 is the plan view of the part shown in FIG. 4;

FIG. 8 shows the relationship between an electrocardiogram signal and a photoplethysmogram signal, which is used to explain the workings of the embodiment of FIG. 3;

FIG. 9 illustrates the PPG waveform obtained using the embodiment of FIG. 3;

FIG. 10 shows the AC components of the PPG waveform of FIG. 9 with DC components removed;

FIG. 11 shows a variation of the embodiment of FIG. 3;

FIG. 12 shows a variation of the embodiment of FIG. 3;

FIG. 13 shows a variation of the embodiment of FIG. 3;

FIG. 14 shows another embodiment, alternative to the embodiment of FIG. 3;

FIG. 15 is the plan view of the embodiment of FIG. 14;

FIG. 16 shows a further embodiment, alternative to the embodiment of FIG. 3;

FIG. 17 is the plan view of the embodiment of FIG. 16;

FIG. 18 shows a variation of the embodiment of FIG. 16;

FIG. 19 illustrates a further embodiment, alternative to the embodiment of FIG. 3;

FIG. 20 is the plan view of the embodiment of FIG. 19;

FIG. 21a is a cross-sectional side view of the embodiment of FIG. 19;

FIG. 21b illustrates an explanation for understanding the functions of the embodiment of FIG. 19;

FIG. 21c illustrates an explanation for understanding the functions of the embodiment of FIG. 19;

FIG. 21d illustrates an explanation for understanding the functions of the embodiment of FIG. 19;

FIG. 22 shows the penetration profile of a particular infrared wavelength with respect to distance between an emitter and an optical sensor;

FIG. 23 shows the penetration profile of a particular red wavelength with respect to distance between an emitter and an optical sensor;

FIG. 24 shows the penetration profile of a particular green wavelength with respect to distance between an emitter and an optical sensor;

FIG. 25 is a penetration profile selected from one of the penetration profiles of FIG. 22 that matches the penetration profiles shown in FIG. 26 and FIG. 27;

FIG. 26 is the penetration profile selected from one of the penetration profiles of FIG. 23 that matches the penetration profiles shown in FIG. 25 and FIG. 27;

FIG. 27 is the penetration profile selected from one of the penetration profiles of FIG. 24 that matches the penetration profiles shown in FIG. 25 and FIG. 26;

FIG. 28 is a variation of the embodiment of FIG. 19;

FIG. 29 is the plan view of the embodiment of FIG. 28;

FIG. 30 shows yet another embodiment, alternative to the embodiment of FIG. 3;

FIG. 31 shows a variation of the embodiment of FIG. 30;

FIG. 32 illustrates how the emitters and optical sensor in all relevant embodiments can be arranged to be directed angularly;

FIG. 33 shows the anatomy of the mouth of a human that may be used with the embodiment of FIG. 34;

FIG. 34 is the schematic diagram of another embodiment, alternative to the embodiment of FIG. 3;

FIG. 35 is an example device in which the embodiment of FIG. 34 may be applied;

FIG. 36 is the absorbance spectrum of HgbA1c, as an example of a blood analyte which can be monitored using the embodiments;

FIG. 37 shows the relationship between absorbance and concentration of the blood analyte of FIG. 36; and

FIG. 38 is the absorbance spectrum of blood glucose, as another example of a blood analyte which can be monitored using the embodiments.

### DETAILED DESCRIPTION OF EMBODIMENTS

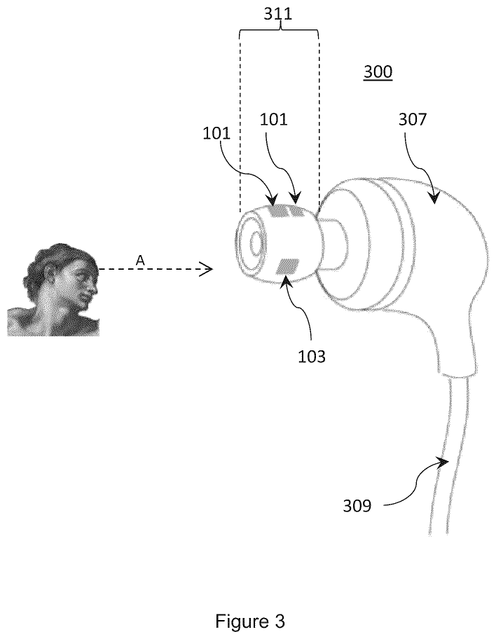

FIG. 3 shows a photoplethysmography (PPG) sensor. The PPG sensor is in the form of an earbud for inserting into an ear canal. The earbud may be part of an earphone or a hearing aid, or may be just a plain earbud without any speaker function.

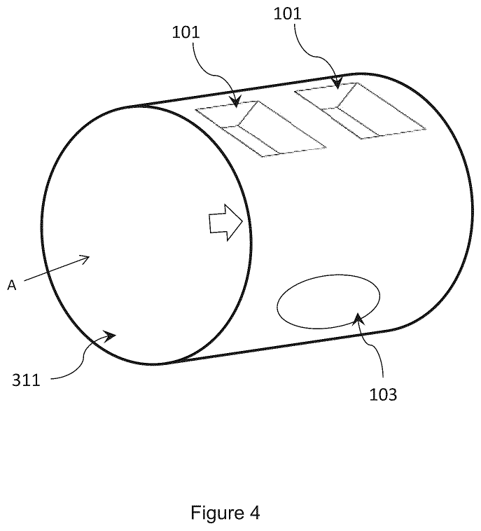

The earbud has an elongate nozzle 311 that extends from a housing. The nozzle 311 is the part of the earbud that is intended to be inserted into the ear canal. FIG. 4 is a schematic illustration of the nozzle 311, which is a stout but elongate body. Along the length of the nozzle 311 are placed two emitters 101 and an optical sensor 103. The directional arrow point labelled “A” points towards the end of the nozzle 311 that inserts into the ear canal. Preferably, the emitters 101 are light emitting diodes (LEDs) and the optical sensor 103 is a photodiode. However, other types of emitters 101 and light detectors can be used.

The first one of the emitters 101 emits in a first wavelength that can be absorbed by the target blood analyte. This means a significant absorption peak in that wavelength is seen in the absorbance spectrum of the blood analyte.

The second one of the emitters 101 emits in second wavelength. However, the absorbance spectrum of the blood analyte does not show any significant absorption peak in the second wavelength. This makes it possible to use the second wavelength to measure the overall background absorption of light by components in the ear canal tissue, i.e. blood, tissue skin and bones, except target analyte.

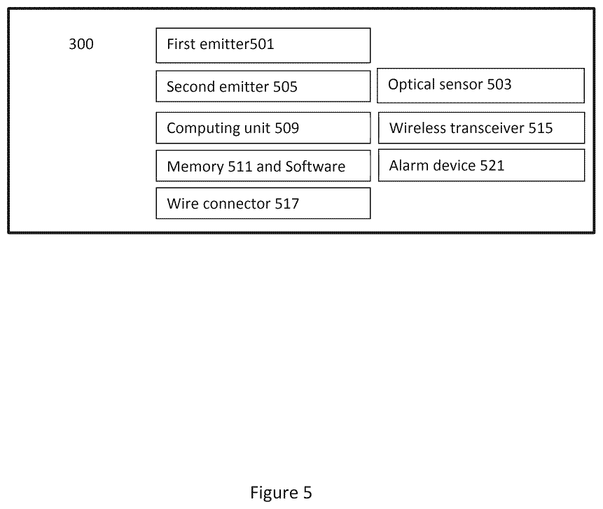

As there is only one optical sensor 103 in this embodiment, the emitters 101 emit sequentially in quick succession, in periods as short as hundreds or even tens of microseconds. Such a fast switching rate allows the pulse to be obtained in each of the two wavelengths concurrently even though just one optical sensor 103 is used. FIG. 5 shows schematically some electronic or functional modules which are may be provided in the PPG sensor 300. As mentioned, there are the first emitter 501, the second emitter 505 and the optical sensor 503. In addition, there is a microprocessor or computing unit 509 and the required memory 511 for operating the PPG sensor 300. Suitable software or firmware for the operation is typically installed in the memory. Optionally, a wireless transceiver 515 is provided for transmitting data to an external device, such as a smart phone or a server. Alternatively, a physical connector 517 is provided for linking the PPG sensor 300 to the external device by a transmission cable 309. Optionally, an alarm device 521 is provided to alert the person that readings produced by the PPG sensor 300 might show a cause for alarm, such as if the level of a blood analyte is too high or too low. The alarm device 521 can be as simple as a sonic device making beeping sounds or a software for transmitting alarm signals to the external device.

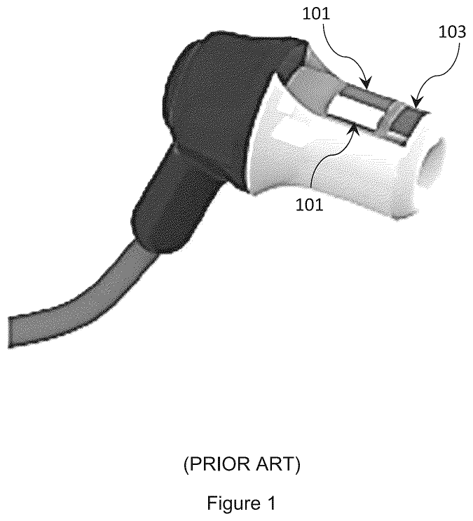

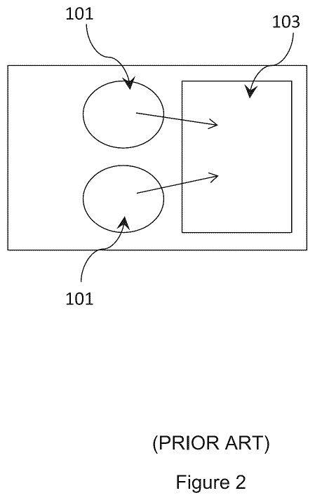

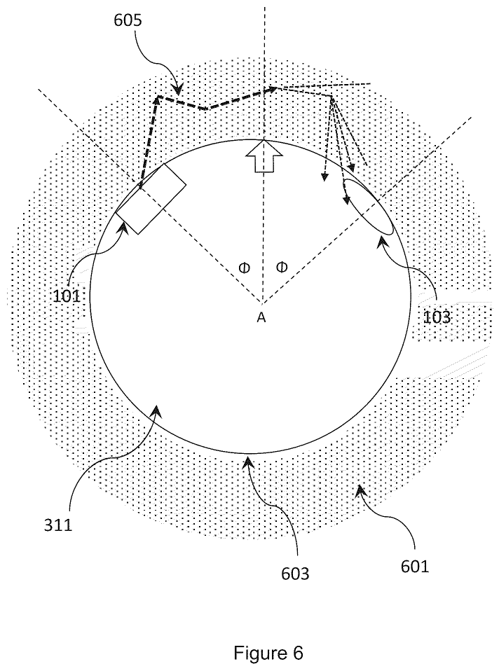

FIG. 6 shows, schematically, the cross-sectional view of the nozzle 311 of FIG. 4 in the direction “A”, revealing that the nozzle 311 is generally round and has a circumference. The white arrow is provided to help the reader oriented to the same point on the earbud drawings when looking at the plan views. The row of two emitters 101 is placed on one side of the nozzle 311 such that the row is parallel and aligned to the axis of the nozzle 311. From the side view, this appears as being placed on a point on the nozzle circumference. In contrast, the optical sensor 103 is placed on another side of the nozzle 311, i.e. placed on another point on the nozzle circumference. The positions of the two emitters 101 overlap in the side view and are therefore not illustrated separately. Therefore, the row of emitters 101 and the optical sensor 103 are mutually and angularly placed away from each other on the nozzle circumference by an angle 2ϕ about the axis of the nozzle 311. This is unlike the prior art in FIG. 1 which provides a flat surface on the nozzle for the placement of all the emitters and optical sensor.

During operation of the PPG sensor, light from the emitters 101 passes into the wall 601 of the ear canal 603. A portion of the light 605 travels and scatters through tissue around the ear canal before exiting from the tissue into the ear canal, and arriving at the optical sensor 103.

The directions of emission of the two emitters 101, which are the same in this embodiment, and the direction of observation of the optical sensor 103 are arranged to point to divergent directions about the nozzle axis (in a nozzle that is not round or oval in the side view, but is made of a curved substrate, this axis would be imaginary). This ensures that light from the emitters 101 has to travel through more tissue before arriving at the sensor. As a result and advantageously, the trajectories are longer than in the prior are in which the emitters and optical sensor are pointed straight towards the ear canal wall, and in the same direction. This leads to greater absorption of the light by the same concentration of components in the tissue, providing higher quantification resolution.

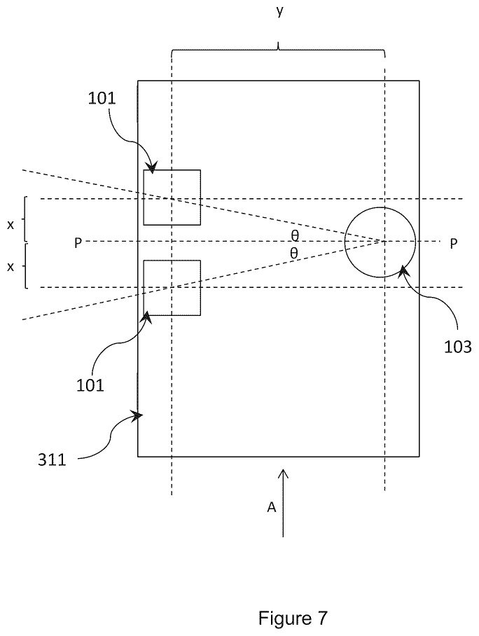

FIG. 7 is the plan view of the nozzle 311 of FIG. 4, in the direction which the white arrow points to. The view is also orthogonal to the direction of the arrow marked “A”. FIG. 7 shows how each of the emitters 101 is placed away from the optical sensor 103 by distance y on an imaginary horizontal plane. The skilled reader would understand that the expression of the distance y is simply made with reference to the planar illustration and, in an actual product, the distance between the emitters 101 and the optical sensor 103 should take into account the curvature of the nozzle circumference.

The emitters 101 are placed adjacent to each other and mutually spaced apart by a distance 2×. This provides a mutual angular displacement of θ about a normal PP in the imaginary horizontal plane.

When in use, the adjacently placed emitters 101 emit into the wall of the ear canal, and the trajectories of the emissions extend through the tissue defining the ear canal before reaching the optical sensor 103. As the trajectories overlap significantly, the depths of penetration of the emissions of both emitters 101 into the tissue are similar. In other words, light from both emitters 101 passes similar amounts of tissue components, including blood and any target analyte. As a result, the absorption readings made in both the wavelengths of the emitters 101 can be compared directly and meaningfully. The absorption readings of the blood analyte in one of the wavelengths can be compared to the absorption readings of background tissue components in the other one of the wavelengths, to quantify the amount of the blood analyte. Calibration, if necessary, is simply a data interpretation method and does not require description here.

The angular difference of 2ϕ between the two emitters 101, as shown in the plan view in FIG. 7, is negligible as long as the trajectories are similar in length and overlap significantly. Preferably and as illustrated, the position of the optical sensor 103 on the length of the nozzle 311 is in between the positions of the two emitters 101. As the skilled reader would appreciate, the value of x is even less significant with a greater y value, as θ becomes more acute. The skilled reader would appreciate that the angular arrangement as illustrated is just schematic, as emitters and optical sensors come in all size and shapes.

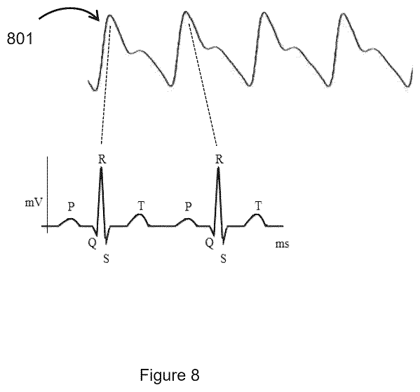

FIG. 8 shows a train of pulse which is typical of PPG signals 801, in which the peaks correlate to the R peaks in the standard PQRST notation of an ECG (electrocardiogram) heart beat pattern. As mentioned, light from the emitter 101 is scattered in all directions inside tissue. A portion of the scattered light is reflected and propagates towards the optical sensor 103. Some of the light is absorbed by blood and tissue in the trajectory of the light towards the optical sensor 103. The amount of blood in the ear pulsates as the heart pumps. In a heartrate cycle, the amount of light absorbed when the ear is pumped full of blood by the heart is more than the amount of light absorbed when the ear is relatively depleted of blood. Therefore, the output of a PPG optical sensor 103 is a signal which has a sinusoidal waveform. That is, the PPG signal has alternating peaks and troughs, like an alternative current (AC).

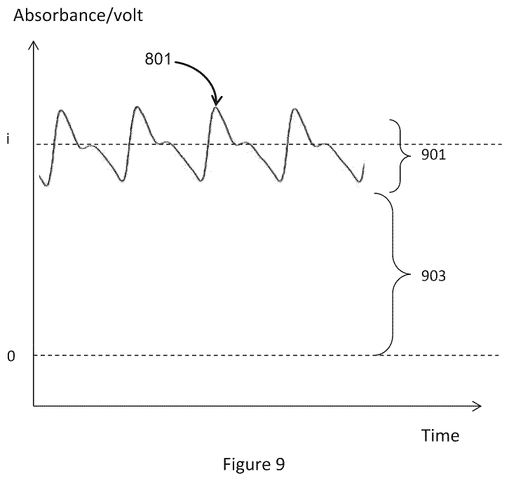

FIG. 9 is an illustration of a typical PPG signal obtained from the heartbeats of a person. The PPG signal 801 comprises an AC part 901 that is superimposed on a larger non-pulsating DC (direct current) part 903. The vertical-axis of FIG. 9 shows absorbance. The AC part 901 is caused by surges of blood in the arteries. The DC part 903, indicated as having a magnitude of y, is caused by relatively unchanging parts of the body, such as skin, tissue, and venous blood, which also absorb the light emitted by the PPG optical sensor 103. Hence, the DC part 903 forms a stable baseline in the PPG signal.

To read data on the analytes in arterial blood, both the signals read by the two emitters 101 are treated to extract their respective AC parts. The AC part 901 is extracted by subtracting the baseline, i.e. the DC part 903, from the PPG signal 801. The extracted AC part 901 is then divided by the DC part 903. In other words, the extracted AC waveform is normalized to its AC/DC ratio.

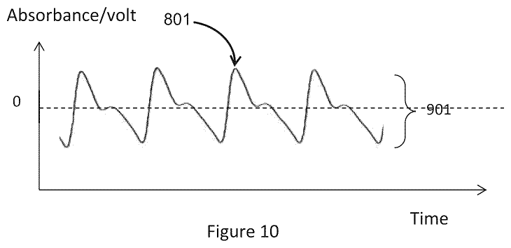

FIG. 10 shows an illustration of a pulse, which is the extracted and normalized AC part 901 centred about 0 volt.

The pulses of the first and second emitters 101 obtained by extraction and normalization of the respective AC parts can be compared with each other by size. The size of the pulse of the first emitter 101 referenced to the size of the pulse of the second emitter 101 provides an indication of the amount of blood analyte in blood. Without normalization of the AC part to the DC part, the pulses cannot be compared as the absolute amplitude of the PPG signal may vary with the quality of the PPG emitter 101 and optical sensor 103, and the size of the merely extracted AC part vary accordingly.

The similar trajectories or transmission paths of light between the two emitters 101 and the optical sensor 103 give a similar effect as providing a standardized cell path in laboratory spectrometry. For completeness, it is mentioned here that the Beer-Lambert law states that the amount of absorbance by a solution is directly proportional to both the concentration of the compound in the solution and the transmission path length through the solution.

$$A = {log}_{10} ( {I}_{0} / I ) = ε cL$$

### Where

A is the measured absorbance (in Absorbance Units (AU))
I₀ is the intensity of the incident light at a given wavelength
I is the transmitted intensity
L is the path length through the sample, and
c is the concentration of the absorbing species.

For each species and wavelength, c is a constant known as the molar absorptivity or extinction coefficient.

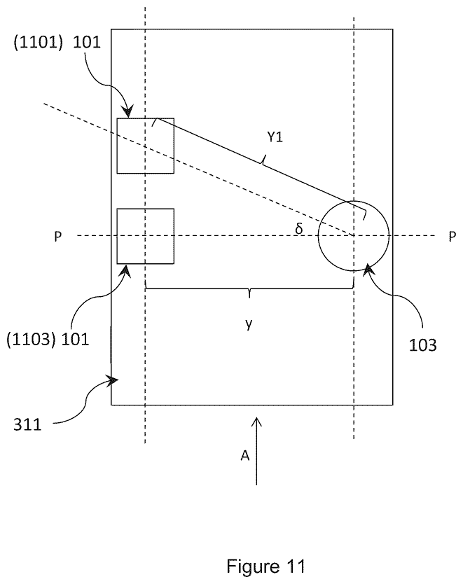

FIG. 11 shows the plan view of a variation of the embodiment of FIG. 7, in which the optical sensor 103 placed nearer the end of the earbud, and is therefore skewed towards one of the emitters 101. This provides that one of the emitters 101 is placed on the same location along the axis as the optical sensor 103, while the other emitter 101 is placed at an angular difference of δ to the optical sensor 103, with reference to the imaginary normal PP. As the two emitters 101 are arranged in a row on one side of the nozzle circumference and are adjacent one to the other, and the optical sensor 103 is on another side of the nozzle circumference, the trajectories of light between both emitters 101 and the optical sensor 103 have a similar distance and overlap significantly.

As the skilled reader would know, the cross-sectional shape of the ear canal seen axially is not a perfect circle but more of an oval with the ends pointing upwardly and downwardly as the person stands, having a typical diameter of about 8 mm to 15 mm. Hence, in some embodiments which are not illustrated here, the cross-sectional shape of the nozzle 311 may be oval in order to fit the shape of the ear canal.

In some embodiment, the cross-section of the nozzle 311 may even be semi-circular or even square in shape, provided that the directions of emission of the two emitters 101 and the direction of observation of the optical sensor 103 are arranged to point to divergent directions about the nozzle axis.

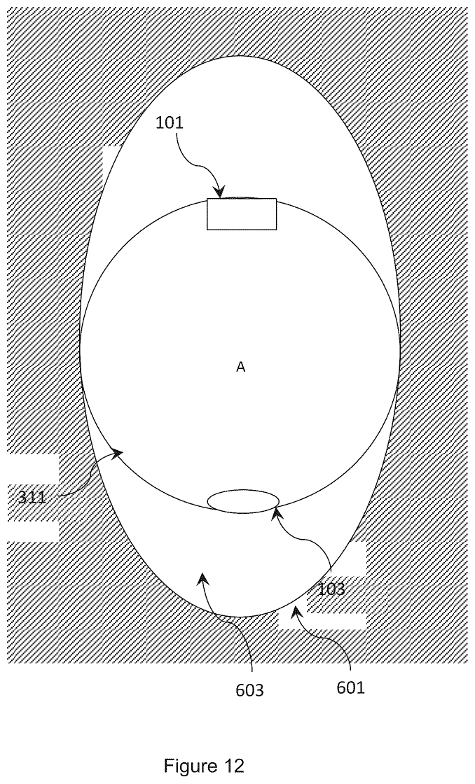

FIG. 12 illustrates another configuration of the placement of the two emitters 101 and the optical sensor 103, wherein the two emitters 101 and the optical sensor 103 are arranged on opposite, vertical sides of the nozzle circumference, such that the emitters 101 emit into the roof of the ear canal 603 and the optical sensor 103 detected light from the floor of the ear canal 603. As a result, light from the emitters 101 have to pass through the ear canal wall 601, and travel through tissue on both sides about the ear canal to exit from the ear canal wall 601 and reach the optical sensor 103. In this case, the earbud has dimensions that the earbud fits into the ear canal in the same positon and orientation every time. The skilled reader would understand that the reverse placement, where the emitters 101 are placed to emit into the floor while the optical sensor 103 is placed to detect light from the roof is within the contemplation of this embodiment.

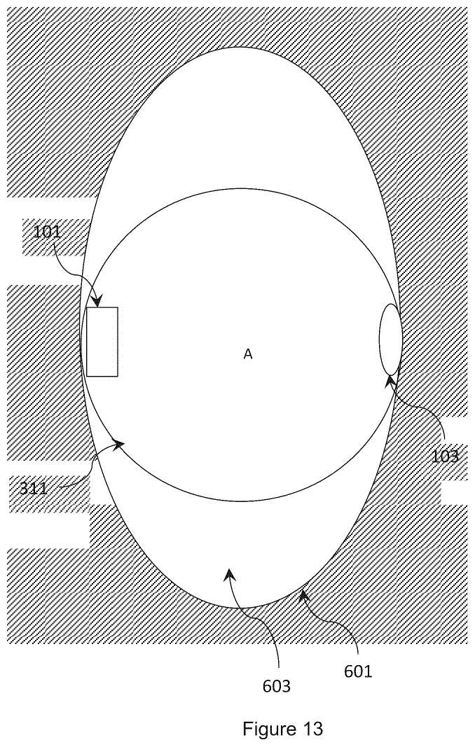

However, as many people have ear canals that are oval in shape, with the ends pointing upwardly and downwardly, the configuration of FIG. 12 provides space for up and down movements of the nozzle 311, which can introduce noise into the readings of the optical sensor 103. Therefore, FIG. 13 illustrates an improved configuration, in which the emitters 101 are placed into contact with one horizontal side of the ear canal wall 601 and the optical sensor 103 placed into contact with the opposite side. This arrangement is likely to provide more stable readings than the configuration shown in FIG. 12, as the up and down movements of the nozzle 311 are unlikely to remove either the emitters 101 or the optical sensor 103 from contact with the wall of the ear canal 603.

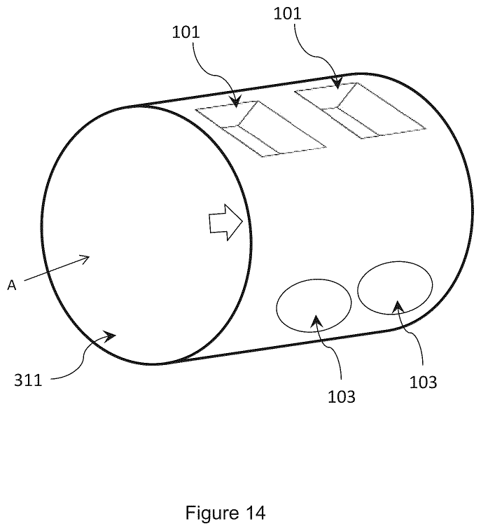

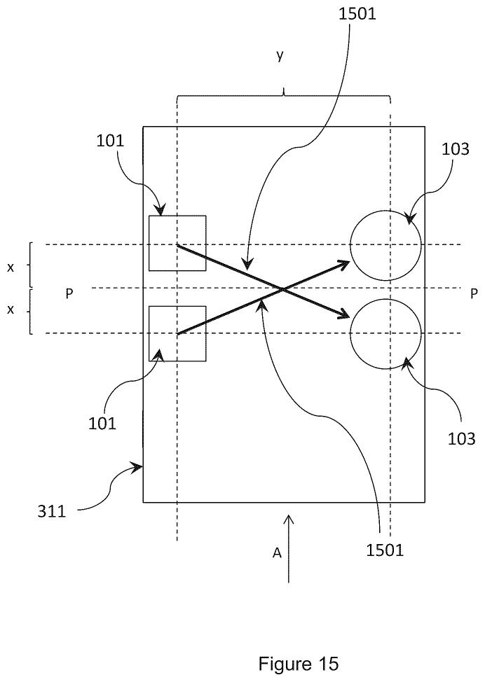

FIG. 14 and FIG. 15 both illustrate another embodiment, in which two optical sensors 103 are provided instead of one. Each of the two optical sensors 103 is covered over by an optical filter which only allows the pre-selected wavelength to pass. Both emitters 101, each emitting in a different wavelength from that of the other, are placed into a row on the same side of nozzle circumference, and in parallel to the nozzle axis. Furthermore, the emitters 101 are placed adjacent to each other. Similarly, both the optical sensors 103 are placed into a row on another side of the nozzle 311, and in parallel to the nozzle axis, and are also adjacent to each other.

Preferably, the positions of the emitters 101 along the length of the nozzle 311 correspond to the respective positions of the optical sensors 103 along the length of the nozzle 311.

In this embodiment, there is no need to alternate between the two emitters 101 sequentially. To improve the likelihood that the trajectories between the emitters 101 and optical sensors 103 overlap and are therefore useable for comparison, the emitters 101 and the optical sensor 103 are paired such that the trajectory of each pair crosses the trajectory of the other pair, as illustrated by the solid arrows 1501 in FIG. 15.

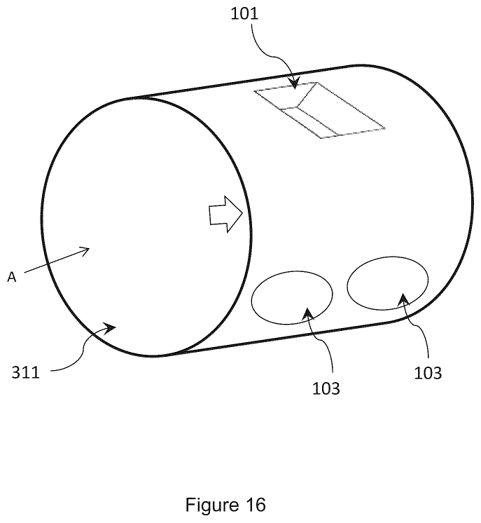

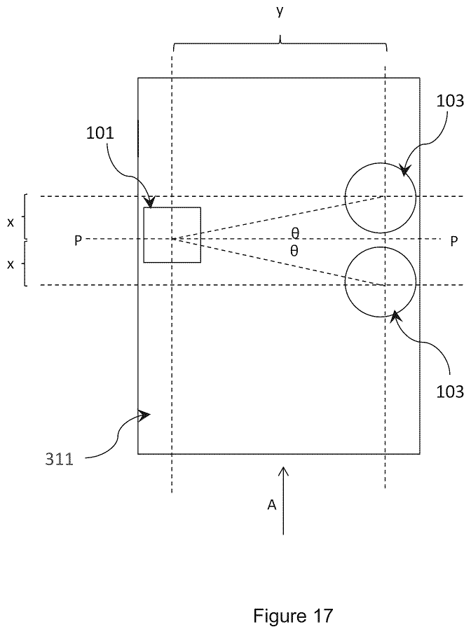

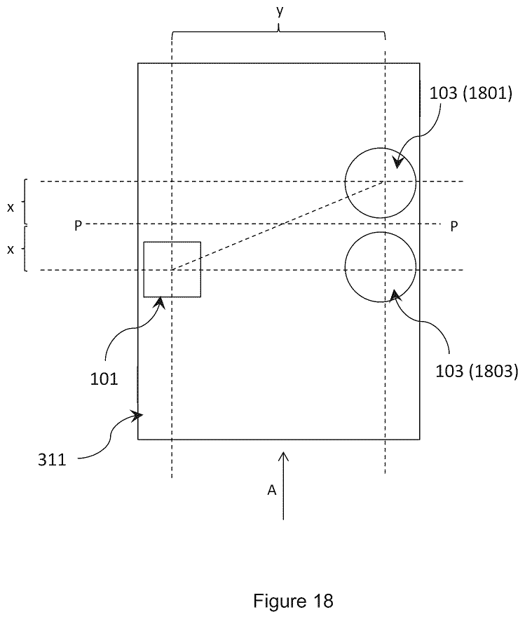

FIG. 16 shows an embodiment in which a single emitter 101 is use as a polychromatic light emitter 101. FIG. 17 is the corresponding plan view, towards the direction of the white arrow in FIG. 16. The single emitter 101 is placed on one side of the nozzle circumference. Two optical sensors 103 each configured to read a different wavelength are placed on another side of the nozzle circumference and arranged into a row that is parallel to the axis of the nozzle 311. Each of the optical sensors 103 is made wavelength-selective by having a suitable wavelength filter placed over the optical sensor 103. As shown in FIG. 17, the two optical sensors 103 are placed equidistant to the two sides about the emitter 101. Thus, the trajectories of light from the emitter 101 to the two optical sensors 103 overlap significantly. However, the skilled reader would understand that, as shown in FIG. 18 which is a variation of FIG. 17, the emitter 101 can be skewed towards one of the optical sensors 103. The skew only creates a small difference in the trajectories which remain similar enough in most practical applications to overlap sufficiently significantly.

However, the embodiment of FIG. 18 can be further improved. Generally, a wavelength that is shorter penetrates less into the depth of the tissue. Hence, in some embodiments, to improve the possibility that the trajectories of light of both wavelengths penetrates to the same depth, the optical sensor 1081 that detects light of a shorter wavelength is placed further from the emitter 101 than the optical sensor 1803 detecting light of a longer wavelength. This further improves the likelihood that the depths of penetration of both trajectories are similar and overlap significantly.

Similarly, turning back to FIG. 11, the emitter 1101 which is further from the optical sensor 103 may emit in the shorter wavelength. The other emitter 1103 which is nearer to the optical sensor 103 may emit in the longer wavelength. Again, this improves the likelihood that the depths of penetration of both trajectories are similar and overlap significantly.

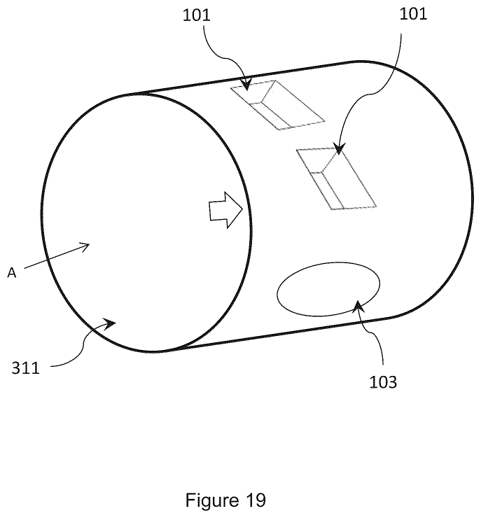

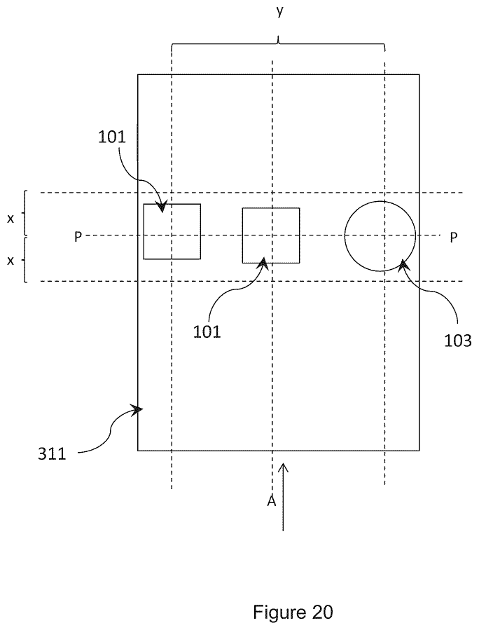

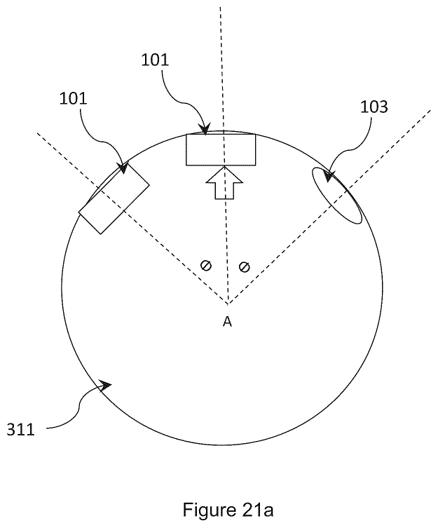

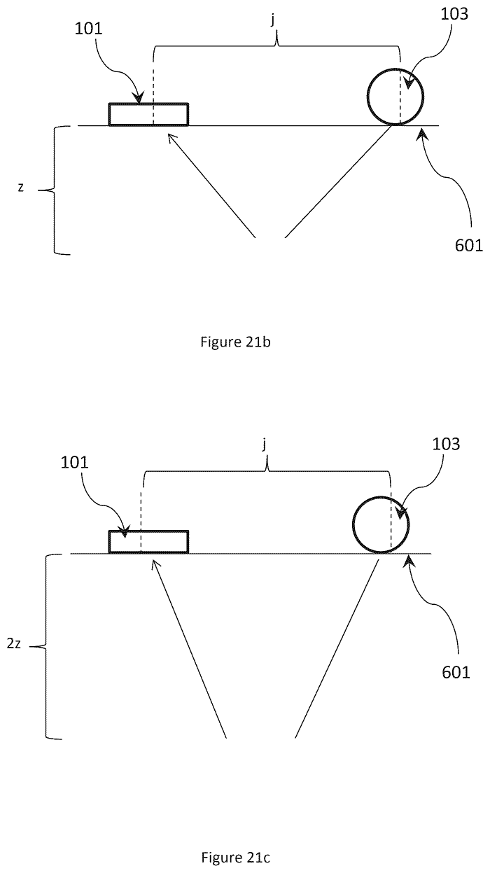

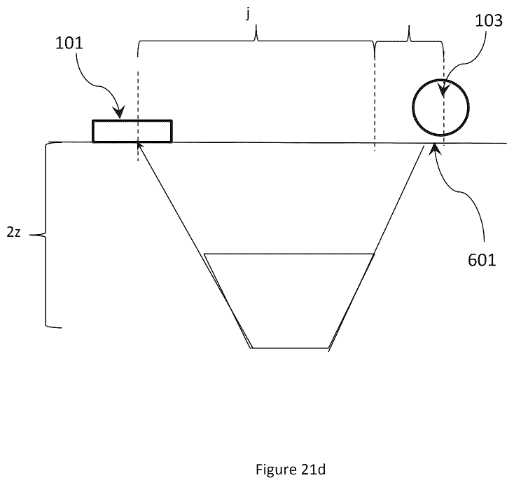

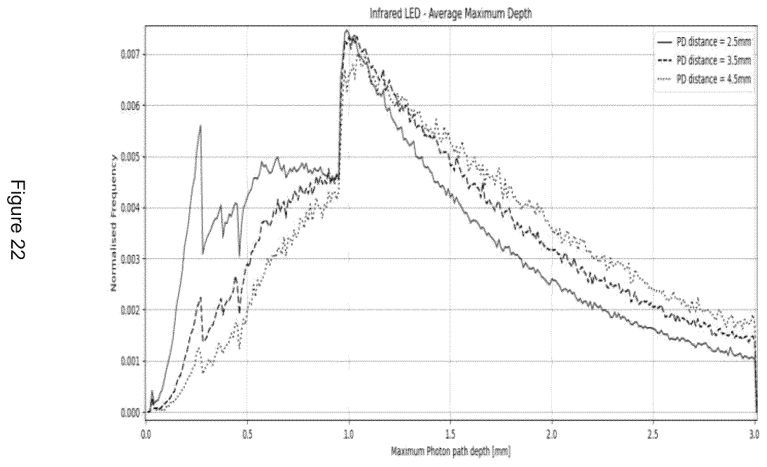

FIG. 19 illustrates a further embodiment, in which there are two emitters 101 and one optical sensor 103. FIG. 20 illustrates the plan of this embodiment, and FIG. 21a illustrates the side view of same embodiment. In this further embodiment, each of all the two emitters 101 and the optical sensor 103 are placed in the same position along the length of the nozzle 311. However, each of all the two emitters 101 and the optical sensor 103 are placed on different sides of the nozzle circumference, and spaced angularly apart about the axis of the nozzle 311. Unlike the prior art shown in FIG. 1, this further embodiment does not require a long nozzle 311 and is therefore more likely to be comfortable to the wearer of the earbud. The emitter 101 that is further from the optical sensor 103 emits in a shorter wavelength. The emitter 101 that is nearer to the optical sensor 103 emits in a longer wavelength.

This provides the possibility that the extent of penetration by the shorter wavelength is deep enough so that sufficient amount of the light is scattered towards the optical sensor 103. This compensate for the natural tendency of the shorter wavelength to penetrate less deeply than the longer wavelength. As a result, the trajectories of the two wavelengths are more likely to overlap and the absorption readings obtained in the two wavelengths become more meaningfully comparable.

FIG. 21b, FIG. 21c, FIG. 21d together explain further how the trajectories are dissimilar for different wavelengths, and how the difference in distance between each of the two emitters and the optical sensor may compensate for the dissimilar trajectories. The skilled reader would appreciate that FIG. 21b, FIG. 21c, FIG. 21d may represent the emitters and optical sensor placed against wall of the ear canal 601, even though the curvature of the ear canal is not depicted, and is shown as flat instead, for ease of illustration.

FIG. 21b illustrates the emitter of the shorter wavelength placed in a distance j from the optical sensor, just as the emitter of the longer wavelength is placed in the same distance j from the optical sensor and as shown in FIG. 21c. FIG. 21d, however, shows the emitter of the shorter wavelength placed in a distance greater than j from the optical sensor, Although the two emitters of in FIG. 21b and in FIG. 21c are placed the same distance from the optical sensor, the penetration depths of the emissions of the two emitters are different, indicated as z in FIG. 21b and as 2z in FIG. 21c. Therefore, the trajectories of light from the two emitters in the tissue and the extent of scattering towards the optical sensor are different. In other words, the volumes of tissue illuminated by light of both wavelengths, which contribute to the respective amounts of detectable scattered light by optical sensor, are different. The part of the tissue that is illuminated is indicated by the shading. Furthermore, the drawings show that that only tissue further down beneath the skin surface contributes to light scattering towards the optical sensor. The shallow part of the tissue immediately below the skin, which is not shaded, does not scatter light from the emitters in any angle could reach the optical sensor.

On the other hand, although the two emitters of in FIG. 21c and in FIG. 21d are placed in different distances from the optical sensor, the penetration depths of the emissions of the two emitters are similar. Therefore, the trajectories of light from the two emitters in the tissue and the extent of scattering towards the optical sensor are similar. In other words, the volumes of tissue illuminated by light of both wavelengths, which contribute to the respective amounts of detectable scattered light by optical sensor, are similar.

Accordingly, the embodiments provide the possibility that PPG sensors having two emitters are no longer limited to using only similar or near wavelengths.

Although the meanings of similar or near wavelengths, or of different or far wavelengths conversely, on the electromagnetic spectrum is subjective, generally the understanding can be assisted by appreciating how different parts of a molecule respond to light of different ranges of wavelengths. In particular, molecular vibrations and stretching respond to infrared light well, i.e. the bending and stretching of bonds between atoms in organic molecules tend to absorb infrared light. Wavelengths that are higher in energy such as ultraviolet end tend to be absorbed by electrons to that the electrons are promoted from one energy level to a higher energy level. Although the change in absorption mechanism from ultraviolet, to visible light and to infrared light is gradual and subtle, once told the molecule or analyte of interest, the skilled reader should generally be able to tell by using standard spectrometry literature whether the absorbance of two selected wavelengths by the molecule are based on the same absorption mechanism. If the absorption mechanisms of the two wavelengths are different, the wavelengths maybe considered dissimilar or far apart.

Accordingly, this embodiment provides the possibility of using two wavelengths that are far apart, one to monitor a blood analyte and the other to monitor background blood components. For example, the wavelength to monitor the blood analyte can be provided by a green light emitting LED, and the wavelength to monitor background blood components can be an infrared emitting LED. Absorption readings obtained using two wavelengths of vastly different electromagnetic spectrum ranges which, in this case, is absorbed by different mechanisms in molecules would not be very meaningfully comparable if the configuration of the prior art in FIG. 1 is used. The present embodiment provides that the depths of penetration and the length of the trajectories of both wavelengths are made more similar such that the absorption readings obtained in both wavelengths become more comparable and may be used for calculation directly in some cases.

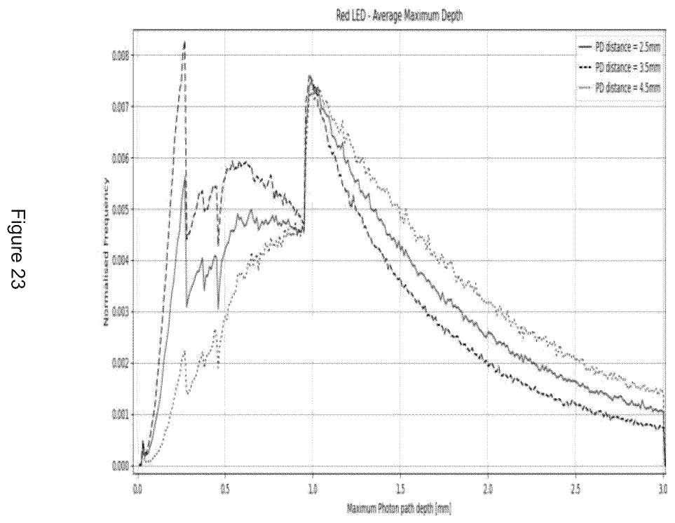

Possibly, the optimal distances of the emitters 101 from the optical sensor 103 can be determined by empirical observation or obtained from mathematical models. FIG. 22 is a chart obtained by Monte Carlo modelling of an LED emitting in a particular wavelength of an infrared light, such as 940 nm. The vertical axis show normalised wavelengths (frequencies) of the infrared light. The horizontal axis shows the depth of penetration into tissue. Each line in the chart is obtained for a different distance between an infrared emitter 101 and an infrared optical sensor 103. In other words, FIG. 22 shows the penetration profile for infrared light into the wall of the ear canal, with respect to the following distances between the emitter 101 and optical sensor 103.
Infrared PD1 (photo diode) at 2.5 mm from the optical sensor 103
Infrared PD2 at 3.5 mm from the optical sensor 103
Infrared PD3 at 4.5 mm from the optical sensor 103

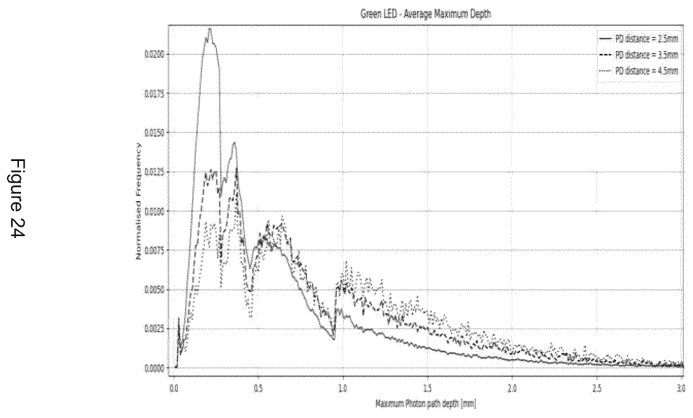

FIG. 23 is a similar chart to FIG. 22, obtained in the same way as FIG. 22 using an LED emitting in red colour at a wavelength of 700 nm and a red light optical sensor:
Red PD1 at 2.5 mm from the optical sensor 103
Red PD2 at 3.5 mm from the optical sensor 103
Red PD3 at 4.5 mm from the optical sensor 103

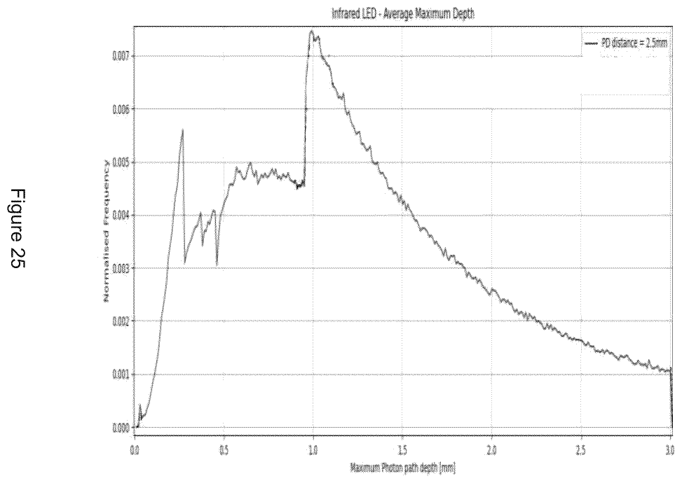

FIG. 24 is a similar chart to FIG. 22, obtained in the same way as FIG. 22 using an LED emitting in green colour, at a wavelength of 400 nm and a green light optical sensor:
Green PD1 at 2.5 mm from the optical sensor 103
Green PD2 at 3.5 mm from the optical sensor 103
Green PD3 at 4.5 mm from the optical sensor 103

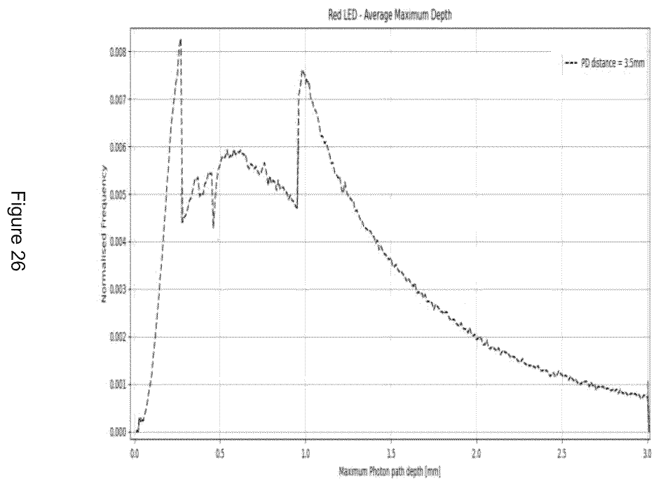

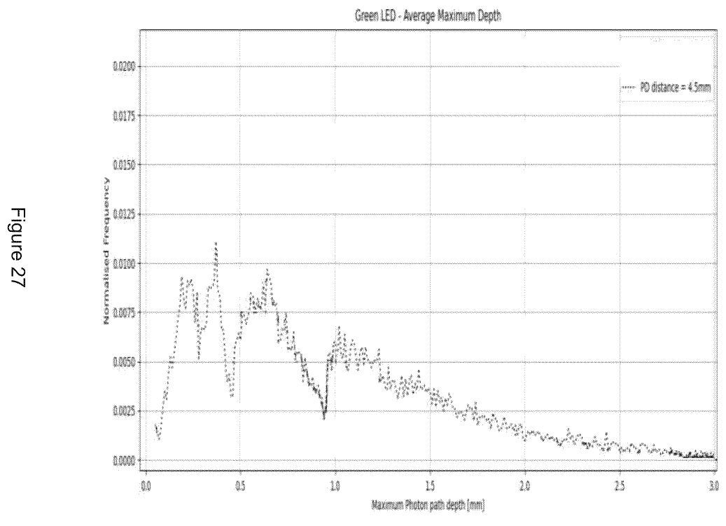

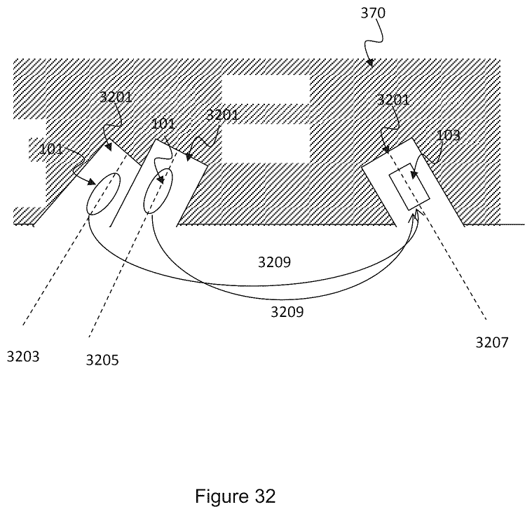

FIG. 25, FIG. 26 and FIG. 27 show the three closest penetration profile of each wavelength taken from the spectra of FIG. 22, FIG. 23 and FIG. 32. As shown, the penetration profiles that are closest are those of the infrared LED placed at 2.5 mm from the optical sensor 103, the emitter 101 of red light placed at 3.5 mm from the optical sensor 103, and the emitter 101 of green light placed at 4.5 mm away the optical sensor 103.

Accordingly, a PPG sensor that has emitters 101 emitting in green and infrared may be produced with the following configuration for improved likelihood that the penetration depths and trajectories of light from both the emitters 101 to the optical sensor 103 overlap significantly, and that the resultant absorption readings may be directly comparable.

| | Wavelength/colour | Distance of emitter 101 |
|---|---|---|
| Emitter 101 | of emitter 101 | from optical sensor 103 |
| 1 | Green | 4.5 mm |
| 2 | Infrared | 2.5 mm |

PPG sensor that has emitters 101 emitting in red and infrared may be produced with the following configuration for improved likelihood that the penetration depths and trajectories of light from both the emitters 101 to the optical sensor 103 overlap significantly, and that the resultant absorption readings may be directly comparable.

| | Wavelength/colour | Distance of emitter 101 |
|---|---|---|
| Emitter 101 | of emitter 101 | from optical sensor 103 |
| 1 | Red | 3.5 mm |
| 2 | Infrared | 2.5 mm |

PPG sensor that has emitters 101 emitting in green and red may be produced with the following configuration for improved likelihood that the penetration depths and trajectories of light from both the emitters 101 to the optical sensor 103 overlap significantly, and that the resultant absorption readings may be directly comparable.

| | Wavelength/colour | Distance of emitter 101 |
|---|---|---|
| Emitter 101 | of emitter 101 | from optical sensor 103 |
| 1 | Green | 4.5 mm |
| 2 | Red | 3.5 mm |

Other colour combinations should have similar arrangements by further Monte Carlo modelling. If the two emitters 101 emit in yellow and orange, for example, the yellow light emitter 101 is placed further from the orange light emitter 101.

A summary of visible and invisible wavelengths is provided below for the befit of the reader.

| Colour | Wavelength interval (nm) |
|---|---|
| Infrared | >700 |
| Red | 700-635 |
| Orange | 635-590 |
| Yellow | 590-560 |
| Green | 560-490 |
| Blue | 490-450 |
| Violet | 450-400 |
| Ultra-violet | <400 |

As the skilled man knows, wavelengths towards the violet end of the spectrum have higher energy and wavelengths towards the red end of the spectrum have lower energy. Wavelengths of higher energy levels are more penetrative into different media than wavelengths of lower energy levels. Hence, blue light is less penetrative than green light, which is in turn less penetrative than red light.

Generally, an emitter 101 of light towards the infrared end of the spectrum and beyond is to be placed nearer the optical sensor 103, and an emitter 101 of light towards the ultraviolet end of the spectrum and beyond is to be placed further from the optical sensor 103.

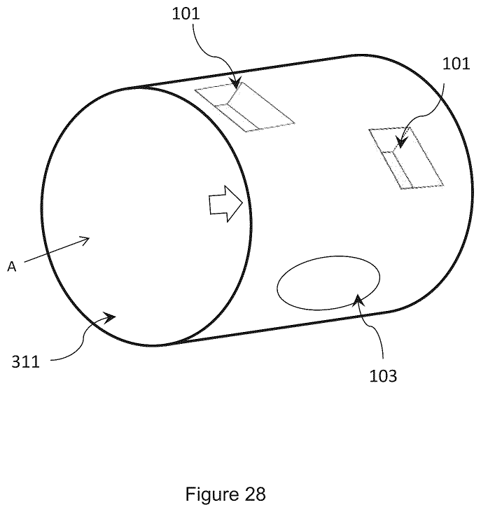

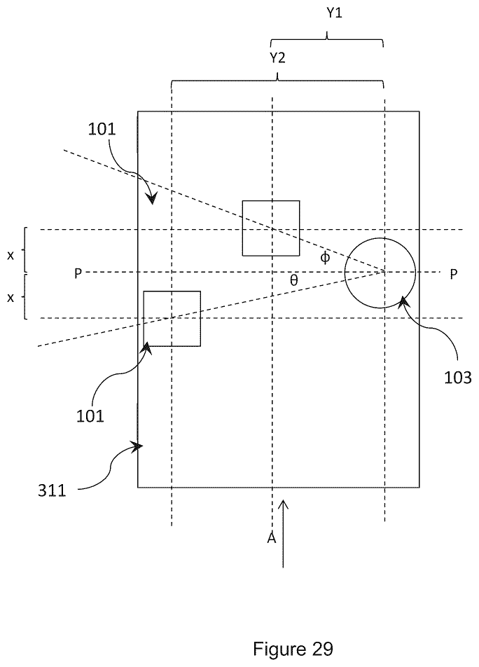

FIG. 28 and FIG. 29 show another embodiment, in which the two emitters 101 and the optical sensor 103 are each placed staggered apart along the length of the nozzle 311, as well as placed on different points on the nozzle circumference. Preferably, the position of the optical sensor 103 on the length of the nozzle 311 is between the positions of the two emitters 101. This embodiment, however, is not as desirable as that of FIG. 19, as the trajectories of the light from the two emitters 101 in FIG. 19 are likely to overlap better. In FIG. 29, one of the emitter 101 is at a distance Y2 in the plan view while the trajectory is an at angle to the optical sensor 103 at an angle θ, while the other emitter 101 is at a distance Y1 and the respective trajectory is at an angle ϕ on the other side of the normal. As explained elsewhere in this description, the skilled man would know that the distance Y1 and Y2 are observed plane and in reality does mean the actual distance as measure on the curved nozzle circumference.

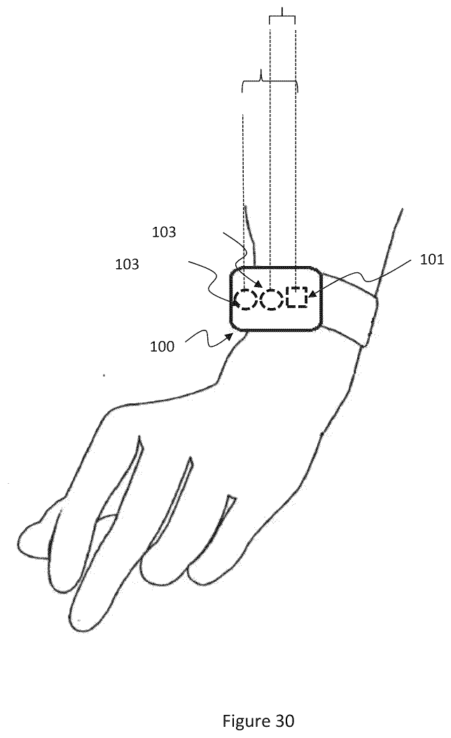

Although most of the embodiments described relate to an earbud to be worn in the ear, this description also contemplates other embodiments such as that shown in FIG. 30, which is a wrist worn PPG device. The PPG device is illustrated as being provided with two emitters 101 and one optical sensor 103. In this case, as the PPG device is not intended to be placed against a concave surface like the wall of the ear canal, there is no curved surface on which the emitters 101 and optical sensor 103 may be spread out and placed. Nevertheless, the emitters 101 can be placed away from the optical sensor 103 in such a way that the emitter 101 emitting in shorter wavelength is placed further from the optical sensor 103, and the emitter 101 emitting in longer wavelength is placed nearer to the optical sensor 103. This ensures that light of the shorter wavelength has to penetrate deeper into the wrist of the user to be scattered towards the optical sensor 103 sufficiently. In this way, the likelihood of the depths of penetration and the length of the trajectories of the light of both wavelengths are likely to overlap significantly, leading to absorption readings that can be compared meaningfully and possibly, directly.

Typically, such PPG sensor is placed near a reflective structure, such as on body parts with thin layer of tissue over a piece of bone or cartilage, like the wrist, the forehead, the shin, the ankle. This allows light from the emitters to be reflected by the bone or cartilage, which improves the scattering of the light towards the optical sensor. Notably, the distance between emitters and sensors should also be large enough in order for the light penetration to deep enough to reach the bone or cartilage in order to be reflected.

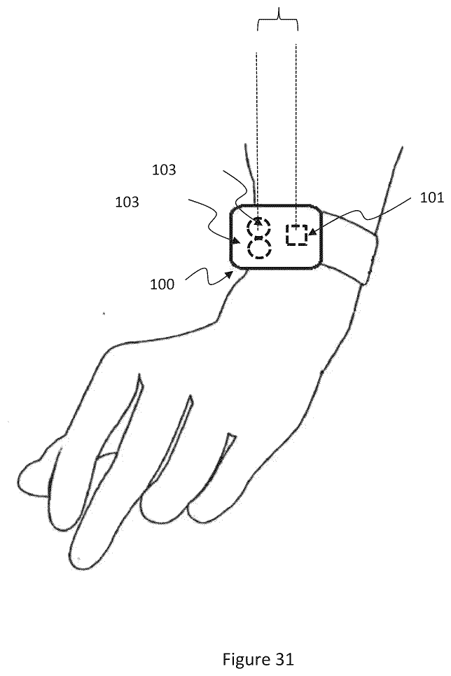

FIG. 31 shows a variation of the embodiment of FIG. 30, in which both emitters 101 are placed at the same distance from the optical sensor 103. Optionally, to provide that light of the emitter 101 emitting in shorter wavelength penetrates more deeply into the wrist, so that the trajectory overlaps significantly with the trajectory of light of the emitter 101 emitting in longer wavelength, both emitters 101 are pointed or directed to emit into the wrist at different angles (although embodiments in which the emitters emit in straight into the wrist and the optical sensor directed to monitor straight from the wrist are also within the contemplation of this embodiment). The emitter 101 of the shorter wavelength emits in a direction that is at a greater angle with the direction which the optical sensor 103 is pointed to. The emitter 101 of the longer wavelength emits in a direction that is at a lesser angle with the direction which the optical sensor 103 is pointed to. This embodiment shows that it is not necessary to place the emitters 101 in different distances to provide the same trajectory or transmission path; it is possible to provide similar trajectories or transmissions path by pointing the emitters 101 in different angles.

FIG. 32 shows one example of how emitters 101 and optical sensors 103 can be pointed in specific directions, wherein the angle to which the emitters 101 and the optical sensor 103 point to may be provided by setting each emitter 101 and optical sensor 103 in a cradle 3201. The cradle 3201 is dug into the surface of a substrate 3703 that is to be applied to the body part. The substrate can be that of the nozzle 311 in FIG. 3, the retainer in FIG. 30 or the underside of the wrist worn device shown in FIG. 30. The cradle 3201 walls and an opening define and direct the cradle axis in a specific direction. Optionally, the base of the cradle 3201 is reflective to better direct all light into the desired direction. As described, the shorter wavelength is pointed in a direction 3203 that forms a greater angle to the direction 3207 to which the optical sensor 103 is pointed, the longer wavelength is pointed in a direction 3205 that forms a lesser angle to the direction 3207 to which the optical sensor 103 is pointed. This provides that emission in the shorter wavelength has to penetrate deeper before enough light is scattered to the optical sensor 103, while emission in the longer wavelength naturally penetrates deeper. Thus, the trajectories 3209 of both wavelengths are made similar, so that both the absorption readings can be directly compared. Many other ways can be used, by physical design or by photonic designs to direct the angle of the light. This methods are known and do no need further elaboration here.

The converse embodiment is also possible (not illustrated), in which an emitter 101 emit in polychromatic light to two optical sensors 103 that are spaced equally from the emitter 101, and on one side of the emitter 101. In such embodiments, each optical sensor 103 has a wavelength filter covering over the optical sensor 103 to provide wavelength selectivity. Each optical sensor 103 is pointed to a different direction. The optical sensor 103 for the shorter wavelength is pointed at away from the polychromatic emitter 101 in a greater angle, while the optical sensor 103 for the longer wavelength is pointed at away from the polychromatic emitter 101 in a lesser angle. Therefore, light of the shorter wavelength has to penetrate deeper before enough of the light is scatter towards the respective optical sensor 103.

This feature of point the emitter is a direction that is divergent from the direction in which the optical sensor is pointed monitor may be used with the classical PPG sensors in which there is only one emitter and only one optical sensor, or in PPG sensors in which there is any number of emitters and any number of optical sensors.

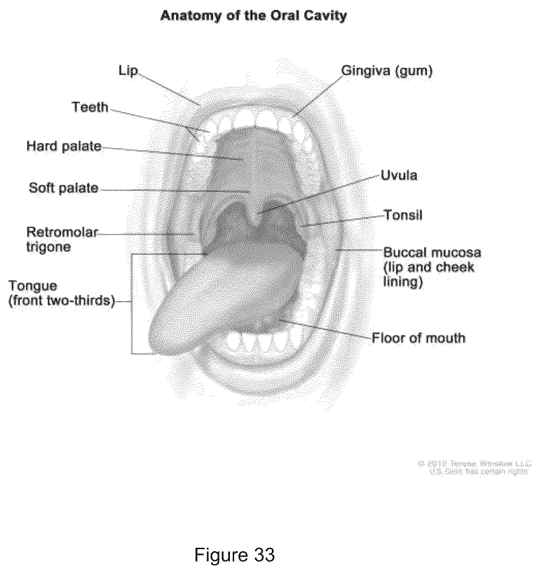

In a further embodiment, the PPG sensor is placed inside a piece of substrate that is shaped to be placed against the floor of the mouth beneath the tongue. An illustration of the anatomy of the mouth is provided in FIG. 33. The illustration is taken from this website: https://www.cancer.gov/publications/dictionaries/cancer-terms/def/oral-cavity. This embodiment is probably not appropriate for wearing over a long period of time, such as over many hours. However, it is possible to wear this embodiment inside the mouth for a few minutes while the PPG optical sensor 103 in the embodiment measures the amount of blood analyte.

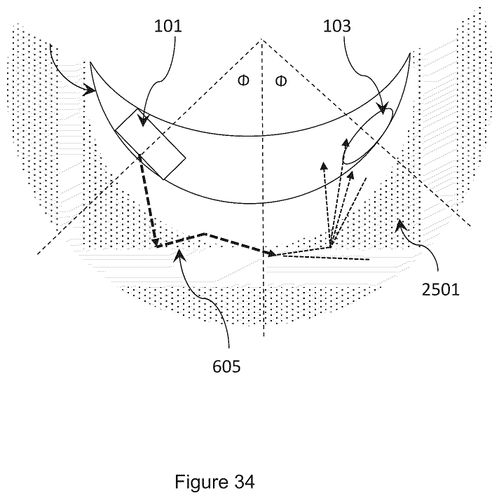

The tissue in the mouth is full of blood and is a suitable location for obtaining measuring analytes in blood using PPG technology. Advantageously, there is no pigmentation on the surface of oral tissue, unlike skin on the outer surfaces of the body which could absorb some of the light from the emitters 101. Also, the structure of the floor of the jaw is slightly concave, and this is convenient for arranging the row of two emitters 101 and the optical sensor 103 to point towards different, divergent directions, respectively. FIG. 34 is a schematic cross-sectional illustration of this embodiment. As shown, the emitters 101 in the embodiment emit directly into the floor 2501 of the mouth and the optical sensor 103 in the embodiment detects scattered light from the floor. The two emitters 101 overlap in this cross-sectional view and cannot be illustrated separately.

The emitters 101 and the optical sensor 103 are mutually and angularly placed away from each other by an angle 2 ϕ about an imaginary origin, such that the direction to which the emitters 101 are pointed to and the direction on which the optical sensor 103 is focused are divergent. Light 605 from the emitters 101 enters into the floor of the mouth and travels in the mouth tissue to reach the optical sensor 103.

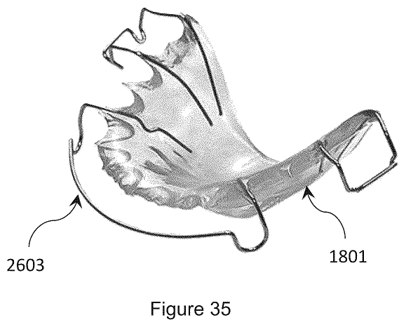

FIG. 35 shows a Hawley Retainer that is used to retain the positions of the lower set of teeth in a person who has just completed a course of orthodontics treatment, also known as braces. The PPG sensor can be made into a form that is similar to the Hawley Retainer. A Hawley Retainer comprises a plastic base 2601 which was melted and cooled in a mould, encasing a part of a metal wire frame 2603 within the plastic. The metal wire frame can be formed to fit onto a set of teeth very tightly, and may be fitted into the same position every time the Hawley Retainer is worn. The difference between the Hawley Retainer and the embodiment is that the base of the embodiment encapsulates the emitters 101 and the optical sensor 103. The skilled reader would note that it is not necessary to make the PPG sensor into such a precise device as a Hawley Retainer. The part of the PPG sensor that fits over the teeth can be modelled on mouth guards that are worn by people in heavy contact sports such as ring boxing.

Alternatively, the roof of the mouth where there is a hard palate can also be used instead of the floor of the mouth beneath the tongue, for these same reasons.

Accordingly, the embodiments include a PPG sensor 300 comprising an earbud nozzle that can be inserted into the ear canal of a user; the nozzle having a curved surface; a first emitter 101, a second emitter 101 and an optical sensor 103 arranged on the curved surface of the nozzle; such that the first emitter 101 and the second emitter 101 are capable of emitting into the wall of the ear canal; the optical sensor 103 is capable of monitoring the light from the wall of the ear canal; wherein the first emitter 101 is placed on a first side on the curvature of the curved surface; the second emitter 101 is placed on a second side on the curvature of the curved surface; the optical sensor 103 is placed on third side on the curvature of the curved surface; and the first emitter 101 and the second emitter 101 are placed to one side of the optical sensor 103 on the curvature of the curved surface.

The embodiments also include a PPG sensor 300 comprising a substrate, the substrate having a surface for being placed against a body part of a user; the surface of the substrate provided with: a) first emitter 101 for emitting light of a first wavelength into the body part, b) second emitter 101 for emitting light of a second wavelength into the body part, and c) at least one optical sensor 103 for sensing light coming from within the body part; the first emitter 101 and the second emitter 101 arranged on the same side of the at least one optical sensor 103, such that the first emitter 101 emits into a first position in the body part; the second emitter 101 emits into a second position in the body part; wherein the trajectory of light from the first position to the at least one optical sensor 103 is greater than the trajectory of light from the second position to the at least one optical sensor 103; and the first wavelength is shorter than the second wavelength.

Also, the embodiments include a PPG sensor 300 comprising a substrate, the substrate having a surface for being placed against a body part of a user; the surface of the substrate provided with: a) a first optical sensor 103 for monitoring light of a first wavelength from the body part; b) a second optical sensor 103 for monitoring light of a second wavelength from the body part; c) at least one emitter 101 for emitting light into the body part in the first wavelength and in the second wavelength, in an emission direction; the first optical sensor 103 and the second optical sensor 103 arranged on the same side of the at least one emitter 101, such that the first optical sensor 103 monitors light in a first position in the body part; the second optical sensor 103 for monitors light in a second position in the body part; the trajectory of light from the first position to the at least one emitter 101 is greater than the trajectory of light from the second position to the at least one optical sensor 103; and the first wavelength is shorter than the second wavelength.

Furthermore, the invention includes a PPG sensor 300 for being placed against a body part, comprising at least a first emitter 101; at least a first optical sensor 103; the first emitter 101 configured to emit in a direction into the body part that forms a divergent angle with the direction in which the first optical sensor 103 is configured to monitor light form the body part.

### Examples of Application of the Embodiments

An example of monitoring one possible analyte in blood, glycated haemoglobin (HgbA1c) is described here below to demonstrate how the embodiment might be used.

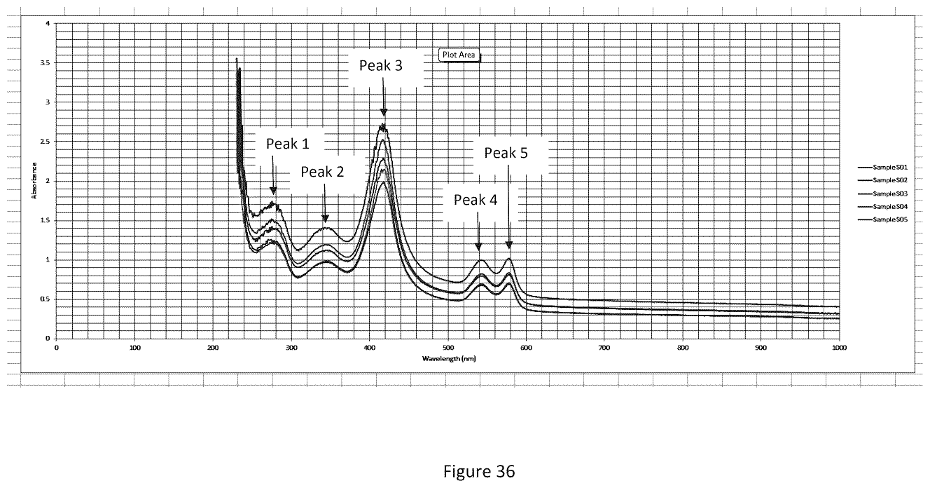

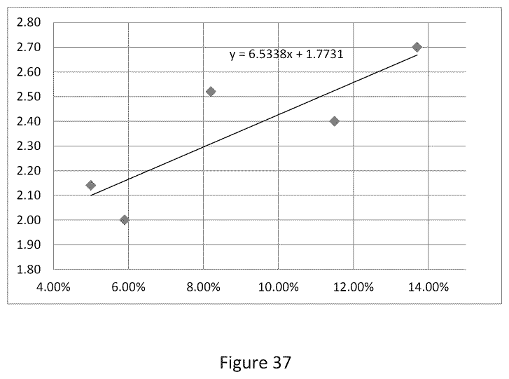

FIG. 36 is an overlay of absorbance spectrums of five samples of human blood. The samples were taken from five people each having a known level of HgbA1c. Peak 3 has maximum absorbance from 415 nm to 420 nm. FIG. 37 is a plot of the absorbance of the five samples at the wavelength of Peak 3.

Table 1 below shows the absorbance data of Peak 3 for each of the blood samples.

TABLE 1

| | HgbA1c concentration | |
|---|---|---|
| Sample no. | (known in advance, | |
| (not in order) | and sorted in order) | Absorbance at 418 nm |
| 3 | 5.00% | 2.14 |
| 4 | 5.90% | 2 |
| 5 | 8.20% | 2.52 |
| 1 | 11.50% | 2.4 |
| 2 | 13.70% | 2.7 |

The absorbance reading at Peak 3 shows a trend with good general correlation with the HgbA1c values.

As can be seen, the plot is generally linear. This shows that it is possible to fit the Peak 3 readings of the five samples into a linear model for quantitative analysis of HgbA1c. In other words, HgbA1c respond to the wavelength of Peak 3 in accordance to the Beer-Lambert law.

Turning now to the second emitter 101, the choice of a red or infrared wavelength as the second wavelength in the second emitter 101 is useful for monitoring the amount of blood content in arteries, as much of the make-up of blood, such as the plasma, is organic. Virtually all organic compounds will absorb infrared wavelengths that correspond in energy to their molecular vibrations. Hence, the second wavelength can be selected from any wavelength starting from 700 nm up. In one preferred embodiment, the second wavelength is 940 nm. This is because LEDs providing light of this wavelength are already commercially available.

Hence a earbud PPG sensor such as that shown in FIG. 19 can be made suitable for monitoring HgbA1c by placing a green light emitter 101 away from the optical sensor 103 about 4.5 mm away on the circumference, and an infrared emitter 101 about 2.5 mm away from the optical sensor 103 on the nozzle circumference.

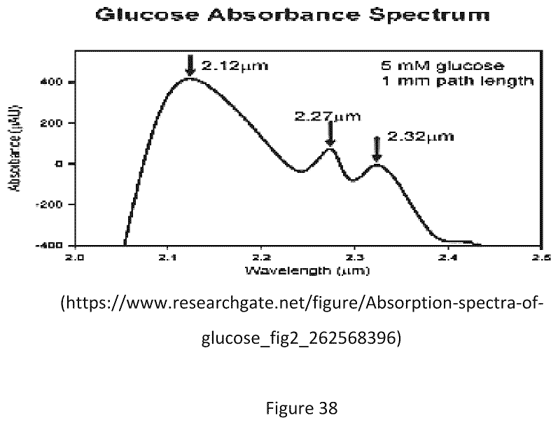

Besides HgbA1c, other analytes in blood can be monitored using this method, such as free glucose in blood, hormones, vitamins, ions and so on. To illustrate this, FIG. 38 shows the spectrum of glucose in the infrared region, showing three distinct peaks. The wavelength of anyone of these peaks can be used in the first emitter 101 as the first wavelength for monitoring glucose level, provided that the absorbance spectrum of glucose does not have any significant absorbance peak in the second wavelength, such as in 940 nm. This example shows that the first wavelength is not restricted to the ultraviolet-visible wavelength range.

While there has been described in the foregoing description preferred embodiments of the present invention, it will be understood by those skilled in the technology concerned that many variations or modifications in details of design, construction or operation may be made without departing from the scope of the present invention as claimed.

For example, although only two emitters 101 have been mentioned in the embodiments, the skilled reader would appreciate that three emitters 101, or more, can be provided in some embodiments. To provide that the trajectories of the light between the respective three emitters 101 to a single optical sensor 103 overlap, the emitter 101 emitting in the shortest wavelength is placed further away from the optical sensor 103, or the emitter 101 emitting in the shortest wavelength is pointed in a greater angle away from the optical sensor 103. Similarly, the emitter 101 emitting in the longest wavelength is placed nearest to the optical sensor 103, or the emitter 101 emitting in the longest wavelength is pointed in a lesser angle away from the optical sensor 103. The third emitter 101 is thus placed in a distance that is between the other two emitters 101, or the third emitter 101 is pointed in a direction that forms an angle that is between the greater angle and the lesser angle formed by the other two emitters 101. Any more emitters 101 can be arranged using the same principle.

Also, the specific distances between the emitters 101 and the optical sensor 103, such as those given as 2.5 mm, 3.5 mm and 4.5 mm should be understood as just examples only, and there is no intention to limit the invention to these numbers and units. The actual distance in a product depends on the size of the product, such as the nozzle 311 in the earbud as described, the curvature of the surface of the nozzle 311, the size and shape of the emitters 101.

1. A PPG sensor comprising
an earbud nozzle that can be inserted into the ear canal of a user;
the nozzle having a curved surface;
a first emitter, a second emitter and an optical sensor arranged on the curved surface of the nozzle; such that
the first emitter and the second emitter are capable of emitting into the wall of the ear canal;
the optical sensor is capable of monitoring the light from the wall of the ear canal; wherein
the first emitter is placed on a first side on the curvature of the curved surface;
the second emitter is placed on a second side on the curvature of the curved surface;
the optical sensor is placed on third side on the curvature of the curved surface; and
the first emitter and the second emitter are placed to one side of the optical sensor on the curvature of the curved surface.

2. A PPG sensor as claimed in claim 1, wherein
the first emitter emits light of a first wavelength;
the second emitter emits light of a second wavelength;
the first wavelength being shorter than the second wavelength; and
the first emitter being further on the curvature of the curved surface from the optical sensor than the second emitter.

3. A PPG sensor as claimed in claim 2, wherein
the absorption mechanism of first wavelength by a target analyte in the wall of the ear canal is different from the absorption mechanism of the second wavelength by the target analyte.

4. A PPG sensor as claimed in claim 1, wherein
the first emitter and the second emitter arranged into a row of emitters;
the row of emitters being placed on the same side on the curvature of the curved surface.

5. A PPG sensor as claimed in claim 1, wherein the optical sensor is a first optical sensor, the PPG sensor further comprising
a second optical sensor;
the second optical sensor arranged on the curved surface of the nozzle; such that
the second optical sensor is capable of monitoring the light from the wall of the ear canal; wherein
the first emitter and the second emitter are placed to one side of the second optical sensor on the curvature of the curved surface.

6. A PPG sensor as claimed in claim 5, wherein
the first optical sensor is capable of monitoring light of the first wavelength;
the second optical sensor is capable of monitoring light of the second wavelength;
the first emitter, the second emitter, the first optical sensor and the second optical sensor arranged such that:
when in the PPG sensor is in use, a first trajectory of light in the wall of the ear canal between the first emitter and the first optical sensor overlaps with a second trajectory of light in the wall of the ear canal between second emitter and the second optical sensor.

7. A PPG sensor as claimed in claim 6, wherein
the first trajectory and the second trajectory cross.

8. A PPG sensor comprising
a substrate, the substrate having a surface for being placed against a body part of a user;
the surface of the substrate provided with:
a) a first emitter for emitting light of a first wavelength into the body part,
b) a second emitter for emitting light of a second wavelength into the body part, and
c) at least one optical sensor for sensing light coming from within the body part;
the first emitter and the second emitter arranged on the same side of the at least one optical sensor, such that
the first emitter emits into a first position in the body part;
the second emitter emits into a second position in the body part; wherein
the trajectory of light from the first position to the at least one optical sensor is greater than the trajectory of light from the second position to the at least one optical sensor; and
the first wavelength is shorter than the second wavelength.

9. A PPG sensor as claimed in claim 7, wherein
the first emitter emits into a first position in the body part by pointing in a first emission direction in the body;
the second emitter emits into a second position in the body part by pointing in a second emission direction in the body;
and the at least one optical sensor is pointed in a sensing direction to sense light coming from within the body part in a sensing direction; wherein
the angle between the first emission direction and the sensing direction is greater than the angle between the second emission direction and the sensing direction.

10. A PPG sensor as claimed in claim 9,
the angle between the first emission direction and the sensing direction is divergent.

11. A PPG sensor as claimed in claim 9, wherein
the angle between the second emission direction and the sensing direction is divergent

12. A PPG sensor as claimed in claim 10, wherein
the surface of the substrate is convex;
the curvature of the convex surface providing the divergence.

13. A PPG sensor comprising
a substrate, the substrate having a surface for being placed against a body part of a user;
the surface of the substrate provided with:
a) a first optical sensor for monitoring light of a first wavelength from the body part;
b) a second optical sensor for monitoring light of a second wavelength from the body part;
c) at least one emitter for emitting light into the body part in the first wavelength and in the second wavelength, in an emission direction;
the first optical sensor and the second optical sensor arranged on the same side of the at least one emitter, such that
the first optical sensor monitors light in a first position in the body part;
the second optical sensor for monitors light in a second position in the body part;
the trajectory of light from the first position to the at least one emitter is greater than the trajectory of light from the second position to the at least one optical sensor; and
the first wavelength is shorter than the second wavelength.

14. A PPG sensor, as claimed in claim 13 wherein
the first position is provided as a first sensing direction;
the second position is provided as a second sensing direction;
the angle between the first sensing direction and the emission direction is greater than the angle between the second sensing direction and the emission direction to provide that the trajectory of light from the emitter to the first optical sensor is longer than the trajectory of light from the emitter to the second optical sensor.

15. A PPG sensor as claimed in claim 14,
the angle between the first sensing direction and the emission direction is divergent.

16. A PPG sensor as claimed in claim 14, wherein
the angle between the second sensing direction and the emission direction is divergent.

17. A PPG sensor as claimed in claim 15, wherein
the surface of the substrate is convex;
the curvature of the convex surface providing the divergence.

18. A PPG sensor for being placed against a body part, comprising
at least a first emitter;
at least a first optical sensor;
the first emitter configured to emit in a direction into the body part that forms a divergent angle with the direction in which the first optical sensor is configured to monitor light form the body part.

19. A PPG sensor for being placed against a body part as claimed in claim 18, further comprising
a second emitter;
the second emitter configured to emit in a direction into the body part that forms a divergent angle with the direction in which the first optical sensor is configured to monitor light form the body part.

20. A PPG sensor for being placed against a body part as claimed in claim 18, further comprising
a second optical sensor;
the first emitter configured to emit in a direction into the body part that forms a divergent angle with the direction in which the second optical sensor is configured to monitor light form the body part.
